(19) United States
(12) Patent Application Publication
(10) Pub. No.: US 2023/0084536 A1
(43) Pub. Date: Mar. 16, 2023

(54) PHARMACEUTICAL COMPOSITIONS FOR IMAGING, DIAGNOSIS AND TREATMENT OF CANCER

(71) Applicant: Jubilant Radiopharma, Kirkland (CA)

(72) Inventors: Vijayaraj Kuniyil Kulangara, Kirkland (CA); Riadh Zriba, Kirkland (CA); Tusharmouli Mukherjee, Yardley, PA (US); Indranil Nandi, Yardley, PA (US); Miriam Ruiz Pena, Kirkland (CA)

(73) Assignee: Jubilant Radiopharma, Kirkland, QC (CA)

(21) Appl. No.: 17/942,911

(22) Filed: Sep. 12, 2022

Related U.S. Application Data
(60) Provisional application No. 63/243,588, filed on Sep. 13, 2021.

(51) Int. Cl. C07K 7/06; A61K 47/22; A61K 47/26; A61K 47/32; A61K 51/04

(52) U.S. Cl. CPC C07K 7/06; A61K 47/22; A61K 47/26; A61K 47/32; A61K 51/041

(57) ABSTRACT

The present invention relates to a pharmaceutical cold kit composition comprising a chelating agent linked to somatostatin receptor binding organic moiety, a stabilizer, a buffer, and a caking agent or bulking agent, wherein the composition is free of sequestering agent. Further, the present invention also relates to the process of preparing reconstituted or radiolabeled solution composition having high in use stability and radiochemical purity and use thereof for diagnostic and/or therapeutic purposes.

### TECHNICAL FIELD

The present invention relates to the pharmaceutical compositions of a chelating agent linked to somatostatin receptor binding organic moiety having high stability and radiochemical purity after labeling with radioisotopes used for diagnostic and/or therapeutic purposes.

### BACKGROUND

Neuroendocrine tumors (NETs) are rare neoplasms. NETs can originate throughout the body from cells of the diffuse endocrine system. More than half of NETs originate from endocrine cells of the gastrointestinal tract and the pancreas, thus being referred to as gastroenteropancreatic NETs (GEP NETs). The only treatment that offers a cure is surgery, however, most patients are diagnosed with metastatic disease, and curative surgery is no longer an option. Thus, early diagnosis, therapy selection, and treatment monitoring are essential for the effective management of NETs, especially the highly prevalent GEP NETs.

[⁶⁸Ga]Ga-DOTATOC injection is a positron emission tomography (PET) radiopharmaceutical agent indicated for the localization of somatostatin receptor positive Neuroendocrine Tumors (NETs) in adult and pediatric patients. The University of Iowa received USFDA approval for Gallium [⁶⁸Ga] radioisotope-based Ga-DOTATOC for IV injection formulation, 18.5-148 MBq (0.5-4.0 mCi/mL).

[⁶⁸Ga]Ga-DOTATOC Injection is a radioactive diagnostic agent for intravenous injection and contains Edotreotide, also known as DOTATOC, an octapeptide, as the active ingredient. The radionuclide ⁶⁸Ga is complexed by the functional chelator DOTA, which acts as a hexadentate chelator with octahedral geometry, coordinating the ⁶⁸Ga to four nitrogen as well as two carboxylic groups. As per the approved labeling instructions, the composition is prepared in a radiopharmacy as a ready-to-use injectable solution to be injected within 3 hours of synthesis. The present manufacturing process is tedious and difficult to implement in other radiopharmacies or hospitals.

Due to the short 68-minutes half-life of ⁶⁸Ga, the currently approved [⁶⁸Ga]Ga-edotreotide Injection cannot be transported to the radiopharmacies, clinics, and hospitals located in distant places as it loses the minimum effective radioactivity required for PET imaging during the transportation. Approved [⁶⁸Ga]Ga-edotreotide Injection contains edotreotide, 18.5 MBq/mL to 148 MBq/mL of [⁶⁸Ga]Ga-DOTATOC at calibration time, and ethanol (10% v/v) in sodium chloride (9 mg/mL) solution. Therefore, there exists an unmet need to develop a stable single and/or multi-dose lyophilized vial kit, which can be easily shipped to a radiopharmacy, where the vial can be radiolabeled by adding the Ga-68 eluted from ⁶⁸Ge/⁶⁸Ga generator or cyclotron, and then distributed to nearby hospitals and other nuclear medicine clinics.

U.S. Pat. No. 9,907,868 discloses the method of complexation of ⁶⁸Ga with chelates, using sequestering agents. According to this patent, the sequestering agent is an essential part of the composition and complexation process with ⁶⁸Ga to neutralize the interfering species leaving the ⁶⁸Ga more free to react with the chelator functionalized molecule. The preferred sequestering agent 1,10-phenanthroline. SomaKit TOC ([⁶⁸Ga]Ga-Edotreotide) approved by the European Medicine Agency comprises a two vial composition wherein: a) the first vial contains edotreotide, 1,10-phenanthroline as sequestering agent, gentisic acid as a stabilizer, mannitol as a diluent; and b) the second vial comprises of formic acid as a buffer, sodium hydroxide and water for injection. The composition comprises 1,10-phenanthroline as a sequestering agent for neutralizing the interfering species. The compound 1,10-phenanthroline is a toxic substance, which should be avoided for human consumption (Material safety data sheet—Scholar Chemistry 2008). Further use of 1,10-phenanthroline can also be hazardous during manufacturing as well.

U.S. Pat. No. 11,045,563 discloses the radiolabeling process of ⁶⁸Ga with chelates however, the composition, process, and kits disclosed require the use of metal inhibitors for improving radiolabeling yields.

U.S. Pat. No. 10,596,276 discloses DOTA complexes with Lu-177 having a high concentration. It requires the use of two different stabilizers and sequestering agents like diethylenetriaminepentaacetic acid (DTPA) in the composition for stabilizing the composition with high concentration.

Patent publication no. WO2020021310 discloses DOTA complexes with Lu-177 having a high concentration. It also requires a sequestering agent like diethylenetriaminepentaacetic acid (DTPA) in the composition for stabilizing the composition having a high concentration.

Another patent publication WO2010092114 discloses the use of at least two buffers for gallium radiolabeling with chelated peptides.

The compositions disclosed in the art require the use of metal inhibitors/sequestering agents for preparing gallium complexes with chelate-linked peptides for minimizing the impact of interfering species. The use of metal inhibitors or sequestering agents in the composition can also affect the complexation of gallium with chelate in addition to inhibiting complexation with interfering species. Some of these sequestering agents can also have toxic effects on human beings. Therefore, there is an unmet need to develop an effective ⁶⁸Ga complexes of edotreotide without the use of any metal inhibitor or sequestering agent which can be readily prepared and easily shipped to a radiopharmacy, where the vial can be radiolabeled by adding the ⁶⁸Ga eluted from a commercially available ⁶⁸Ge/⁶⁸Ga generator or cyclotron.

The inventors of the present invention were able to design a novel cold-kit formulation comprising a single stabilizer, single buffer, and without the use of sequestering agent. The prepared cold-kit or lyophillized formulation can be successfully radio labelled with Gallium in radiopharmacy. The prepared compositions as per the present invention have acceptable radiochemical purity initially, during shelf life, and during transportation or time lag before administering to the patients.

### SUMMARY

It is an object of the present invention to provide stable radiolabeled compositions of an imaging agent or therapeutic agent.

It is an object of the present invention to provide stable compositions of radioisotope labeled complexes with a protein, peptide, antibodies, or small molecules.

It is another object of the present invention to provide stable compositions of radioisotope labeled complexes with a chelating agent linked to somatostatin receptor binding organic moiety.

It is an object of the present invention to provide stable compositions of radioisotope labeled complexes with edotreotide or its pharmaceutically acceptable salts thereof.

It is an object of the present invention to provide stable compositions of ⁶⁸Ga labeled complexes with edotreotide or its pharmaceutically acceptable salts thereof, wherein the composition is free of sequestering agent(s).

It is an object of the present invention to provide stable compositions of ⁶⁸Ga labeled complexes with edotreotide or its pharmaceutically acceptable salts thereof, wherein the composition is free of sequestering agents.

It is an object of the present invention to provide stable compositions of ⁶⁸Ga labeled complexes with edotreotide or its pharmaceutically acceptable salts thereof, wherein the composition comprises a single buffer.

It is an object of the present invention to provide stable ready to dilute compositions of ⁶⁸Ga labeled complexes with edotreotide or its pharmaceutically acceptable salts thereof, wherein the composition comprises a single stabilizer.

It is an object of the present invention to provide stable ready to dilute compositions of ⁶⁸Ga labeled complexes, wherein the composition comprises edotreotide or its pharmaceutically acceptable salts thereof, a buffer, a caking agent or bulking agent, and a stabilizer.

It is an object of the present invention to provide stable compositions of ⁶⁸Ga labeled complexes, wherein the composition comprises edotreotide or its pharmaceutically acceptable salts thereof, ⁶⁸Ga eluted from ⁶⁸Ge/⁶⁸Ga generator or cyclotron produced, a buffer, a caking agent or bulking agent, a stabilizer and hydrochloric acid.

It is an object of the present invention to provide single dose and multiple dose stable compositions of ⁶⁸Ga labeled complexes of edotreotide or its pharmaceutically acceptable salts thereof.

It is an object of the present invention to provide a process of preparing single vial or multiple vial compositions with vial size ranging from 1-30 mL of the present invention.

It is an object of the present invention to provide a process of radiolabeling single vial or multiple vial compositions with vial size ranging from 10-30 mL of the present invention.

The present invention concerns any of the following items:

In one aspect of the present invention, a pharmaceutical composition comprising: a chelating agent linked somatostatin receptor binding organic moiety, a stabilizer, a buffer, and a caking agent or bulking agent, wherein the composition is free of sequestering agent.

In yet another aspect of the present invention, a pharmaceutical composition comprising: edotreotide or its pharmaceutically acceptable salts thereof; a stabilizer selected from the group consisting of ascorbic acid, sodium bisulfite, gentisic acid, glutamate, metabisulfite, monothioglycerol, propyl gallate, sulfite sodium, tocopherol alpha, thioglycolate, formaldehyde sulfoxylate sodium, histidine, melatonin; a buffer selected from the group consisting of sodium acetate, sodium succinate, tartrate, citrate, formate, HEPES buffer, lactate, TRIS, phosphate, nitrate, phosphate, borate, bicarbonate, carbonate, a zwitterionic buffer, morpholine propanesulphonic acid, dextrose, lactose, tartaric acid, arginine and combinations thereof; a caking agent or bulking agent selected from the group consisting of sodium chloride, gelatin, mannitol, inositol, sorbitol, polyethylene glycol, and polyvinyl pyrrolidone (PVP) and combinations thereof, wherein the composition is free of sequestering agent.

In another aspect of the present invention, a chelating agent is selected from DOTA, NOTA, TETA, DTPA, NTA, EDTA, D03A, NOC, DOTAGA, MED, HBECC, NODAGA, DFO, EDTA, 6SS, B6SS, PLED, TAME, YM103, and H₂dedpa.

In another aspect of the present invention, the pharmaceutical composition is a kit comprising a single vial or multiple vial kit with vial size ranging from 1-30 mL.

In another aspect of the present invention, the pharmaceutical composition is a kit comprising a single vial or multiple vial kit with vial size ranging from 10-30 mL.

In another aspect of the present invention, the multiple vial kit comprises two vials.

In another aspect of the present invention, the multiple vial kit comprises three vials.

In another aspect of the present invention, the multiple vial kit comprises four vials.

In another aspect of the present invention, the pH of the composition ranges from about 3-8.

In another aspect of the present invention, the two-vial kit comprises: a) a first vial comprising a chelating agent linked somatostatin receptor binding organic moiety, a stabilizer, and a caking agent or bulking agent; and b) a second vial comprising a buffer.

In another aspect of the present invention, the three-vial kit comprises: a) a first vial comprising a chelating agent linked somatostatin receptor binding organic moiety, a stabilizer, and a caking agent or bulking agent; b) a second vial comprising a buffer; and c) a third vial comprising a hydrochloric acid solution.

In another aspect of the present invention, the four-vial kit comprises: a) a first vial comprising a chelating agent linked somatostatin receptor binding organic moiety, a stabilizer, and a caking agent or bulking agent; b) a second vial comprising a buffer; c) a third vial comprising hydrochloric acid; and d) a fourth vial being an empty vacuumed vial.

In another embodiment according to the present invention, the pharmaceutical cold kit composition is radiolabeled with the desired radioisotope and is used in the field of nuclear medicine applications for radionuclide imaging and therapy.

In yet another aspect of the present invention, the pharmaceutical composition is radiolabeled with a radioisotope selected from the group consisting of copper-64, gallium-68, gallium-67, gallium-66, lutetium-177, yttrium-86, yttrium-90, indium-114, indium-111, scandium-47, scandium-44, scandium-43, zirconium-89, bismuth-213, bismuth-212, actinium-225, lead-212, rhenium-188, rhenium-186, and rubidium-82.

In another aspect of the present invention, the radioisotope is gallium-68, lutetium-177 or actinium-225.

In another aspect of the present invention, the radiochemical purity of the radiolabeled composition is not less than 95 percent.

In another aspect of the present invention, the initial radiochemical purity of the radiolabeled composition is at least 97 percent, or at least 99 percent or at least 100 percent.

In another aspect of the present invention, the composition comprises less than 3 percent of ⁶⁸Ga in colloidal form.

In another aspect of the present invention, the composition comprises less than 2 percent of Gallium 68 (III) ion.

In another aspect of the present invention, there is provided a method of preparing a lyophilized single vial cold-kit composition comprises the steps of: a) adding a buffer to deoxygenated water for injection and mixing until dissolution; b) adding an antioxidant and caking agent or bulking agent to the solution and mixing until complete dissolution; c) adding edotreotide or its pharmaceutically acceptable salts thereof; d) mixing the solution for dissolution and bringing to the final QS target with deoxygenated water for injection to obtain bulk pre-lyophilized solution; and e) lyophilizing the final solution; wherein the composition is free of sequestering agents and wherein the composition is further reconstituted or radiolabeled with the desired radioisotope.

In another aspect of the present invention, there is provided a method of preparing a single vial kit radiolabeled composition comprises: a) optionally adding 0.5 mL of water to a lyophilized vial; b) adding 1-10 mL ⁶⁸Ga in hydrochloric acid solution to the solution of step (a) after 1-2 minutes; c) heating the vial for 5-30 minutes at a temperature ranging from 60-120° C. and cooling for 2-10 minutes; and d) diluting the vial to the desired volume with saline.

In another aspect of the present invention, the method of preparing a two vial kit radiolabeled composition comprises: a) adding 1-10 mL ⁶⁸Ga in hydrochloric acid solution to vial one containing lyophilized composition; b) adding buffer solution of vial two to the vial one solution of step (a); c) heating the vial for 5-30 minutes at a temperature ranging from 60-120° C. and cooling for 2-10 minutes; and d) diluting the vial to the desired volume with saline.

In another aspect of the present invention, the method of preparing a two vial kit radiolabeled composition comprises: a) adding 1-10 mL ⁶⁸Ga in hydrochloric acid solution to vial one containing lyophilized composition; b) adding buffer solution of vial two to the vial one solution of step (a); c) heating the vial for 5-30 minutes at a temperature ranging from 60-120° C. and cooling for 2-10 minutes; and d) diluting the vial with saline at a volume of 4-14 ml, wherein the saline is sodium chloride present at a concentration of about 7 mg/mL-11 mg/mL.

In another aspect of the present invention, the method of preparing a two vial kit radiolabeled composition comprises: a) adding 1-10 mL ⁶⁸Ga in hydrochloric acid solution to vial one containing lyophilized composition; b) adding buffer solution of vial two to the vial one solution of step (a); c) heating the vial for 5-30 minutes at a temperature ranging from 60-120° C. and cooling for 2-10 minutes; d) diluting the vial with saline at a volume of 4-14 ml, wherein the saline is sodium chloride present at a concentration of about 7 mg/mL-11 mg/mL; wherein the edotreotide is present in the solution at a concentration of about 3 μg/mL-4 mg/mL.

### DETAILED DESCRIPTION

Positron Emission Tomography (PET) and Single-Photon Emission Computerized Tomography (SPECT) agents are important tools for imaging, diagnosing, and treating various diseases like cancer. PET and SPECT agents should be bound to the ligands, which further bind to the specific receptors in the body. Once PET and SPECT agents are injected into the body, the same can be imaged using techniques like Positron Emission Tomography (PET) or Single-Photon Emission Computerized Tomography (SPECT), and finally a report is generated which shows the diseased state for further treatment. Therefore, there is a need to develop novel and stable compositions of such agents to administer radioactive substances effectively to track the diseased location in the body for appropriate imaging, diagnosing, and treatment. The present invention can be more readily understood by reading the following detailed description of the invention and included embodiments.

As used herein, the term ‘imaging’ refers to techniques and processes used to create images of various parts of the human body for diagnostic and treatment purposes within digital health. Such imaging techniques and processes include X-ray radiography, Fluoroscopy, Magnetic resonance imaging (MRI), Computed Tomography (CT), Medical Ultrasonography or Ultrasound Endoscopy Elastography, Tactile Imaging, Thermography Medical Photography, and nuclear medicine functional imaging techniques e.g. Positron Emission Tomography (PET) or Single-Photon Emission Computed Tomography (SPECT).

As used herein, the term ‘Positron Emission Tomography (PET)’ refers to a functional imaging technique that uses radioactive substances known as radiotracers or radionuclides to visualize and measure changes in metabolic processes, and in other physiological activities including blood flow, regional chemical composition, and absorption.

As used herein, the term ‘Single-Photon Emission Computed Tomography (SPECT)’ refers to anuclear medicine tomographic imaging technique using gamma rays and provides true 3D information. Obtained technical information is typically presented as cross-sectional slices through the patient, but can be freely reformatted or manipulated as required. The technique requires the delivery of a gamma-emitting radioisotope (a radionuclide) into the patient, normally through injection into the bloodstream. A marker radioisotope is generally attached to a specific ligand to create a radio ligand, which facilitates binding it to certain types of tissues or receptors. This allows the combination of ligand and radiopharmaceutical to be transported and bound to a region of interest in the body, wherein the ligand concentration is assessed by a gamma camera.

As used herein, the term ‘diagnosis’ refers to a process of identifying a disease, condition, or injury from its signs and symptoms. A health history, physical exam, and tests, such as blood tests, imaging, scanning, and biopsies, can be used to help making a diagnosis.

As used herein, the term ‘dose’ refers to the radioactivity dose of a radionuclide required to perform imaging in a subject. The dose of a radionuclide to be administered to the subject ranges from about 0.01 MBq to 10,000 MBq.

As used herein, the term ‘radionuclide’ or ‘radioisotope’ refers to an unstable form of a chemical element that releases radiation as it disintegrates and becomes more stable. Radionuclides can occur in nature or can be generated in a laboratory. In medicine, they are used in imaging tests and/or in treatment.

As used herein, the term ‘eluant’ refers to the liquid or the fluid used for selectively leaching out the daughter radioisotopes from the generator column.

As used herein, the term ‘eluate’ refers to the radioactive eluant after the acquisition of daughter radioisotope from the generator column.

As used herein, the term ‘generator’ or “radioisotope generator” refers to a hollow column inside a radio-shielded container. The column is filled with an ion exchange resin and radioisotope loaded onto the resin.

As used herein, the term ‘metal inhibitors’ or ‘sequestering agents’ are used as a synonymous word, which refers to the agents that is used to bind or chelate the undesirable contaminating metal ions together, which are interfering and/or competing with the chelation of the radioactive metal and form a complex to improve the quality of the product. Further, these agents are co-chelating agents, which are capable of inactivating contaminating metals other than Ga-68 or other radioisotopes of interest, without interfering with the chelation between Ga-68 and said a chelating agent linked somatostatin receptor binding organic moiety. Some examples include sugars, monosaccharides, or derivatives of monosaccharides such as tetracetose, pentacetose, hexacetose, tetrose, pentose, hexose, D-mannose, D-fructose, and derivatives; and/or disaccharides and their derivatives such as maltose and its derivatives; and/or polysaccharides and their derivatives such as dextrins, cyclodextrins, sulfated sugars, cellulose and derivatives, glycine and other chelating amino acids, crown ethers, and nitrogen crown ethers, heterocyclic organic compound e.g. 1,10-phenantroline, 2,2′-Bipyridine, calixarenes, polydentate chelator e.g. proteins, polysaccharides, and polynucleic acids, natural chelating agents e.g. catechins, tannin, porphyrin, in general, linear or macrocyclic chelating agents (for example podands or kryptands).

As used herein, the term ‘lyophilization’ refers to the freeze drying or cryodesiccation process in which water is removed from a product after it is frozen and placed under a vacuum, allowing the ice to change directly from solid to vapor without passing through a liquid phase by sublimation.

As used herein, the term ‘cold-kit’ refers to a large amounts of reagent sufficient for labeling single or multiple doses of radiopharmaceuticals. Further, cold kit-based radiolabeling is considered a closed procedure, consisting of preparation of a sterile radiopharmaceutical through the addition of a sterile eluate to a sterilized close vial containing a set of sterile, lyophilized ingredients via a system closed to the atmosphere.

As used herein, the term ‘stabilizers’ or ‘antioxidants’ are used as a synonymous word, which refers to an agent which protects organic molecules against radiolytic degradation. These substances or agents prevent oxygen from reacting with other compounds that are susceptible to oxidation. The stabilizer may be able to scavenge radicals, which may be generated, for example, when the radionuclide emits a gamma ray and the gamma ray cleaves a bond between the atoms of organic molecules, thereby forming radicals. Hence, the stabilizer can avoid or reduce that radicals undergo other chemical reactions, which might lead to undesired, potentially ineffective or even toxic molecules.

Radionuclide generator according to the present invention is selected from ⁹⁹Mo/^{99m}Tc, ⁹⁰Sr/⁹⁰Y, ⁸²Sr/⁸²Rb, ¹⁸⁸/¹⁸⁸Re, ⁶⁸Ge/⁶⁸Ga ⁴²Ar/⁴²K, ⁴⁴Ti/⁴⁴Sc, ⁵²Fe/^{52m}Mn, ⁷²Se/⁷²As, ⁸³Rb/^{83m}Kr; ¹⁰³Pd/^{103m}Rh, ¹⁰⁹Cd/^{109m}Ag, ¹¹³_{Sn/}^{113m}In, ¹¹⁸Te/¹¹⁸Sb, ¹³²Te/¹³²I, ¹³⁷Cs/^{137m}Ba, ¹⁴⁰Ba/¹⁴⁰La, ¹³⁴Ce/¹³⁴La, ¹⁴⁴Ce/¹⁴⁴Pr, ¹⁴⁰Nd/¹⁴⁰Pr, ¹⁶⁶Dy/¹⁶⁶Ho, ¹⁶⁷Tm/^{167m}Er, ¹⁷²Hf/¹⁷²Lu, ¹⁷⁸W/¹⁷⁸Ta, ¹⁹¹Os/^{191m}Ir, ¹⁹⁴Os/¹⁹⁴Ir, ²²⁶Ra/²²²Rn and ²²⁵Ac/²¹³Bi.

Radioisotope according to the present invention is selected from copper-64, gallium-68, gallium-67, gallium-66, lutetium-177, yttrium-86, yttrium-90, indium-114, indium-111, scandium-47, scandium-44, scandium-43, zirconium-89, bismuth-213, bismuth-212, actinium-225, lead-212, rhenium-188, rhenium-186, technetium-99m and rubidium-82. Preferably, the radioisotope according to the present invention is Gallium-68, Lutetium-177, and Actinium-225. The gallium-68 according to the present invention can be produced by a generator or cyclotron. Cyclotron production can be manual, semi-automatic, or fully automatic process. Cyclotron Gallium-68 production can be based on a solid target or liquid target. High radioactivity can be achieved using cyclotron based production. Zn-68 can be used as a target for producing Gallium-68 using cyclotrons.

In an embodiment according to the present invention, a pharmaceutical cold-kit composition comprising: a chelating agent linked to somatostatin receptor binding organic moiety, a stabilizer, wherein the stabilizer is selected from the group consisting of ascorbic acid, gentisic acid, sodium bisulfite, tocopherol alpha, thioglycolate, a buffer and a caking agent or bulking agent, wherein the composition is free of sequestering agents and wherein the composition is further radiolabeled with the desired radioisotope.

In an embodiment according to the present invention, a pharmaceutical cold-kit composition comprising: edotreotide or its pharmaceutically acceptable salt thereof; a stabilizer selected from the group consisting of ascorbic acid, sodium bisulfite, gentisic acid, glutamate, metabisulfite, monothioglycerol, propyl gallate, sulfite sodium, tocopherol alpha, thioglycolate, formaldehyde sulfoxylate sodium, and melatonin; a buffer selected from the group consisting of sodium acetate, sodium succinate, tartrate, citrate, formate, HEPES buffer, lactate, TRIS, phosphate, nitrate, phosphate, borate, bicarbonate, carbonate, a zwitterionic buffer, morpholine propane sulphonic acid, dextrose, lactose, tartaric acid, arginine and combinations thereof; and a caking agent or bulking agent selected from the group consisting of sodium chloride, gelatin, mannitol, inositol, sorbitol, polyethylene glycol, and polyvinyl pyrrolidone (PVP); polyethylene glycol, and polyvinyl pyrrolidone (PVP) and combinations thereof; wherein the composition is free of sequestering agent and wherein the composition is further radiolabeled with desired radioisotope.

In an embodiment according to the present invention, an imaging agent can be a fluorescent agent. Fluorescent agents include oregon green fluorescent agents like oregon green 514, oregon green 488, alexafluor fluorescent agents like alexafluor 647, alexafluor 488 and the like, fluorescein, and related analogs; bodipy fluorescent agents, including but not limited to bodipy F1, bodipy 505; rhodamine fluorescent agents, including but not limited to tetramethylrhodamine, and the like; dylight fluorescent agents, including but not limited to dylight 680, dylight 800, and the like, CW 800, texas red, phycoerythrin, and others.

In another embodiment according to the present invention, the buffer is selected from sodium acetate, sodium succinate, tartrate, citrate, formate, HEPES buffer, lactate, TRIS, sodium ascorbate, phosphate, nitrate, phosphate, borate, bicarbonate, carbonate, a zwitterionic buffer, morpholine propane sulphonic acid, dextrose, lactose, tartaric acid, and arginine. The amount of buffer in the composition ranges from about 1 mg to 200 mg, preferably from about 5 to 100 mg. The percentage amount of buffer in the composition ranges from 5-90% w/w of the total weight of the composition.

In an embodiment according to the present invention, the caking agent or bulking agent is selected from sodium chloride, gelatin, mannitol, inositol, sorbitol, polyethylene glycol, and polyvinyl pyrrolidone (PVP). The amount of caking agent or bulking agent in the composition ranges from about 1 mg to 100 mg, preferably the caking agent or bulking agent is present in an amount from about 1 mg to 50 mg, preferably from about 1 mg to 20 mg. The percentage amount of caking agent or bulking agent in the composition ranges from about 3% to about 80% of the total weight of the composition.

In an embodiment according to the present invention, the stabilizer or antioxidant is selected from ascorbic acid, sodium bisulfite, gentisic acid, glutamate, metabisulfite, monothioglycerol, propyl gallate, sulfite sodium, tocopherol alpha, thioglycolate, formaldehyde sulfoxylate sodium, and melatonin. The amount of stabilizer in the composition ranges from about 0.01 mg to 10 mg, preferably the stabilizer is present in an amount from about 1 mg to 10 mg, from about 1 mg to 7 mg, from about 1 mg to 5 mg. The percentage amount of stabilizer in the composition ranges from about about 0.01 to about 40% of the total weight of the composition.

In an embodiment according to the present invention, the radioactivity of the composition ranges from 0.0001 MBq to 10,000 MBq.

In yet another embodiment according to the present invention, the ligand comprises a peptide, protein, antibody, or a small molecule. The peptide can be a somatostatin receptor binding organic moiety or prostate specific membrane antigen binding moiety selected from edotreotide, pentetreotide, PSMA-11, PSMA-I&T, PSMA-617, DCFPyL, PSMA-1007, MIP-1404 or their pharmaceutically acceptable salts thereof. The pharmaceutically acceptable salt comprises acetate, phosphate, sulfate, sulfonate, hydrochloride, hydrobromide, tartrate, and succinate, preferably acetate salt. The ligand can be further linked to a chelating agent. The amount of ligand in the composition ranges from 0.01 μg to 30,000 μg, preferably the ligand is present in an amount 10 μg to 500 μg, 10 μg to 300 μg, 10 μg to 200 μg, 10 μg to 100 μg, and 10 μg to 70 μg.

In an embodiment according to the present invention, the chelate complexes with a radioisotope. The chelates according to the present invention for somatostatin receptor binding organic moiety and prostate specific membrane antigen binding moiety is selected from the group consisting of DOTA, DOTATATE, DOTATOC, DOTANOC, NOTA, TETA, DTPA, NTA, EDTA, D03A, NOC, DOTAGA, HBED, HBECC, NODAGA, DFO, EDTA, 6SS, B6SS, PLED, TAME, YM103, and H2dedpa.

The term ‘about’ as used herein refers to a measurable value such as a parameter, an amount, a temporal duration, and the like, and is meant to encompass variations of and from the specified value, in particular variations of ±10% or less, preferably ±5% or less, more preferably ±1% or less, and still more preferably ±0.1% or less of and from the specified value, such variations are appropriate to perform in the disclosed invention. It is to be understood that the value to which the modifier ‘about’ refers is itself also specifically, and preferably, disclosed.

In another embodiment, the composition can be lyophilized, solution, suspension, or an emulsion.

In another embodiment, the lyophilized composition is reconstituted with ⁶⁸Ga solution and may further be diluted with a suitable pharmaceutically acceptable diluent before administering/infusing into the patient. The infusion rate for IV injection ranges from 0.01 mL/second to 5 mL/second.

In an embodiment according to the present invention, the composition can be a single vial or multiple vial composition. The multiple vial composition comprises two vials, three vials, or four vials. The single vial composition comprises a peptide bound to a chelate, a stabilizer, a buffer, and a caking agent or bulking agent. The two vials kit composition comprises: a) a first vial comprising a peptide bound to chelate, a stabilizer, and a caking agent or bulking agent; and b) a second vial comprising a buffer. The three vial kit composition comprises: a) a first vial comprising a peptide bound to chelate, a stabilizer, and a caking agent or bulking agent; b) a second vial comprising a buffer; and c) a third vial comprising a hydrochloric acid solution. The four vial kit composition comprises: a) a first vial comprising a peptide bound to chelate, a stabilizer, and, a caking agent or bulking agent; b) a second vial comprising a buffer; c) a third vial comprising hydrochloric acid solution; and d) a fourth vial comprising an empty vacuumed vial.

In another embodiment, the kit may further comprise additional accessories required for diluting, re-constituting, or radiolabeling the lyophilized composition with radionuclide eluate and optional dilution with a biocompatible solution for administering to the patient. The accessories may comprise syringes, vial adapters, tubing, and one or more additional empty vials.

In another embodiment there is provided, a process of preparing a single vial and two vial lyophilized composition/kit of edotreotide or its pharmaceutically acceptable salts thereof. In one embodiment, there is provided a method of preparing a single vial lyophilized composition comprises adding a buffer to deoxygenated water for injection and mixing until complete dissolution; adding an anti-oxidant and caking agent or bulking agent to the solution and mixing until complete dissolution; adding edotreotide or its pharmaceutically acceptable salts thereof and mixing the solution for dissolution and bringing to the final quantum sufficit target with deoxygenated water for injection to obtain bulk pre-lyophilized solution and then lyophilizing. In an additional embodiment, a caking agent or bulking agent is added to the composition. In another embodiment, there is provided a process of preparing a two vial composition and kit comprises: 1) Vial A—Adding a stabilizer to deoxygenated water and mixing until complete dissolution, then adding a caking agent or bulking agent to the solution and mixing until complete dissolution, then adding edotreotide or its pharmaceutically acceptable salts thereof and mixing the solution for dissolution and bringing to final quantum sufficit (QS) target with deoxygenated water for injection to obtain bulk pre-lyophilized solution and then lyophilizing; 2) Vial B—buffer preparation—Preparing sodium citrate 0.1M pH 5: Mixing 350 mL of citric acid 0.1M with 650 mL of sodium citrate 0.1M for preparing 1 L of sodium citrate 0.1M pH 5. Inert gases can be used for deoxygenation like argon, nitrogen, and helium. The amount of oxygen in the lyophilized vial is less than 5 percent, preferably less than 2 percent, less than 1 percent, less than 0.5 percent, or less than 0.1 percent. The amount of oxygen in pre-lyophilized bulk solution is maintained up to 5 ppm, preferably less than 3 ppm, less than 1 ppm, and less than 0.5 ppm.

In another embodiment, the process of radiolabeling a single vial kit is provided. The process of radiolabeling comprises: a) optionally, adding about 0.5 to 2 mL of water to a lyophilized vial; b) adding 1-5mL of the radioactive eluate in the vial after about 1-5 minutes; c) heating the vial for about 5-30 minutes at a temperature of about 60-120° C., preferably between 95-105° C. and cooling for about 1-20 minutes; d) diluting the vial with saline solution to the desired volume. Alternatively, radiolabeling can be performed by microwave. In another embodiment, the process of radiolabeling a two-vial kit is provided. The process comprises: a) adding gallium-68 eluate solution in Vial A comprising lyophilized peptide bound to chelate, stabilizer, and a caking agent or bulking agent; b) adding a buffer to solution in Vial B; c) the final product is radiolabeled by using any one selected method: ci) Adding Gallium-68 in hydrochloric acid solution to Vial A. Adding buffer solution of Vial B after 1-2 minutes and heating the vial for about 15 minutes and cooling for 10 minutes, and then diluting it to the desired volume with saline; cii) Adding buffer solution of Vial B to Vial A. Adding Gallium-68 in hydrochloric acid solution after 1-2 minutes. Heating the vial for 15 minutes and cooling for about 10 minutes, and then diluting to the desired volume with saline; and ciii) Mixing Gallium-68 in hydrochloric acid solution with the buffer solution of Vial B and then adding the mixture to Vial A. Heating the vial for 15 minutes and cooling for about 10 minutes, and then diluting to the desired volume with saline. Alternatively, radiolabeling can be performed by microwave.

The radiochemical purity of the composition is found to be not less than 95% for both single-vial and two-vial composition kits.

In another embodiment, the composition comprises less than 3 percent of Gallium 68 in colloidal form, preferably less than 2 percent, more preferably less than 1 percent, and even more preferably less than 0.5 percent.

In another embodiment, the composition comprises 2 percent of Gallium 68 (III) iron, preferably less than 1 percent, more preferably less than 0.5 percent, and even more preferably less than 0.2 percent.

In another embodiment, the pH of the lyophilized composition ranges from about 3 to 7, preferably between 4 to 8.

In another embodiment, the initial pH of the lyophilized composition ranges from about 4 to 8, preferably from about 4 to 7.

In another embodiment, the pH level of the lyophilized composition is within 0.5 pH units, preferably 0.2 pH units of an initial pH level after storage at 25±2° C. and 60±5% relative humidity for 12 months.

In another embodiment, the pH level of the lyophilized composition is within 0.5 pH units, preferably 0.2 pH of an initial pH level after storage at 40±2° C. and 75±5% relative humidity for 6 months.

In another embodiment, the pH of the radiolabeled composition ranges from 2 to 8, preferably between 2 to 6.

In another embodiment, the initial pH of the radiolabeled composition ranges from 2.5 to 5.

In another embodiment, the pH level of the radiolabeled composition is within 0.5 pH units of an initial pH level after storage at 25±2° C. and 60±5% relative humidity for 3 hours.

In another embodiment, the pH level of the radiolabeled composition is within 0.2 pH units of an initial pH level after storage at 25±2° C. and 60±5% relative humidity for 3 hours.

In another embodiment, the lyophilized vial comprises less than 5 percent of oxygen in the headspace, preferably less than 3 percent, more preferably less than 1 percent, even more preferably less than 0.5 percent.

In another embodiment, the lyophilized vial is depyrogenated and terminally sterilized.

In another embodiment, there is provided a method of administering a radiolabeled composition of edotreotide or its pharmaceutically acceptable salts thereof to a subject for diagnosis and/or treatment of Neuroendocrine tumor comprises drawing a radiolabeled edotreotide composition from the vial into the syringe, injecting the composition into the patient, wherein the composition comprises 0.01 μg to 30,000 μg of edotreotide, a buffer, a stabilizer, a caking agent or bulking agent, a radionuclide, and wherein the composition is free of sequestering agent.

In another embodiment, there is provided a method of imaging a subject for diagnosis of neuroendocrine tumor comprises drawing a radiolabeled edotreotide or its pharmaceutically acceptable salts thereof composition from the vial into the syringe, injecting or infusing the composition into the patient, wherein the composition comprises 0.01 μg to 30,000 μg of edotreotide, a buffer, a stabilizer, a caking agent or bulking agent, a radionuclide, and wherein the composition is free of sequestering agent.

In another embodiment according to the present invention, the pharmaceutical cold kit composition is radiolabeled with the desired radioisotope and is used in the field of nuclear medicine applications for radionuclide imaging and therapy.

In another embodiment, there is provided a method of treating a subject suffering from neuroendocrine tumor comprises drawing a radiolabeled edotreotide composition from the vial into the syringe, injecting or infusing the composition into the patient, wherein the composition comprises 0.01 μg to 30,000 μg of edotreotide, a buffer, a stabilizer, a caking agent or bulking agent, a radionuclide, and wherein the composition is free of sequestering agent.

In another embodiment according to the present invention, a process of preparing single vial or multiple vial with sizes ranging from 1-30 mL pharmaceutical compositions of edotreotide is provided.

In another embodiment according to the present invention, a process of preparing single vial or multiple vial with sizes ranging from 10-30 mL pharmaceutical compositions of edotreotide is provided.

In another embodiment according to the present invention, the radiolabeled composition is automatically infused by an infusion device like an infusion system or infusion pump controlled via a controller. The administration via infusion system comprises the automatic calculation of the infusion time, and flow rate depending upon the radioactivity or dose to be administered to the patient. The dose can be calculated based on patient parameters selected from body habitus, height, weight, body mass, body surface area, lean body mass, body mass index, thoracic or abdominal circumference diseased condition of the patient, age, sex, pregnancy, waist circumference, and body fat. In an additional embodiment, the infusion system may comprise a dose calibrator to calculate the amount of radioactivity before administration to the patient. The infusion system comprises a controller, input screen, monitoring screen, dose calibrator, waste container, generator, pump, tubing, printer, memory, and diluents.

In another embodiment according to the present invention, the radiolabeled dose can be prepared by dispensing system automatically. The dispensing system may comprise a controller, input screen, monitoring screen, dose calibrator, waste container, pump, pliers, tubing, and memory. The dispensing system can be controlled from a distance so that the operator may have minimum exposure to radiation.

In another embodiment according to the present invention, the compositions can be used for imaging, diagnosis, and treatment of cancer.

In another embodiment according to the present invention, the radiochemical purity of the radiolabeled composition is not less than 95%, not less than 96%, not less than 97%, not less than 98%, not less than 99%, not less than 100%.

In another embodiment according to the present invention, the ratio of stabilizer to edotreotide ranges from 20:1 to 400:1, preferably the ratio of stabilizer to edotreotide ranges from 50:1 to 300:1, more preferably the ratio of stabilizer to edotreotide ranges from 60:1 to 120:1.

In another embodiment according to the present invention, the radiochemical purity of the radiolabeled composition at initial (time zero) immediately after radiolabeling is at least 95%, preferably at least 96%, preferably at least 97%, more preferably 98%, more preferably 99% and even more preferably 100%. Since the half-life of gallium 68 is 68 minutes, therefore, it is important to have initial radiochemical purity as high as possible so that it can be easily transported to hospitals, and patients at other locations after radiolabeling to get sufficient radioactivity for use.

In another embodiment according to the present invention, reconstituted pharmaceutical composition comprising: from 0.045 mg/vial to 0.055 mg/vial of edotreotide or its pharmaceutically acceptable salt thereof; from 2 mg/vial to 5 mg/vial of a stabilizer selected from the group consisting of ascorbic acid, sodium bisulfite, gentisic acid, glutamate, metabisulfite, monothioglycerol, propyl gallate, sulfite sodium, tocopherol alpha, thioglycolate, formaldehyde sulfoxylate sodium, histidine, and melatonin; from 45 mg/vial to 90 mg/vial of a buffer selected from the group consisting of sodium acetate, sodium succinate, tartrate, citrate, formate, HEPES buffer, lactate, TRIS, phosphate, nitrate, phosphate, borate, bicarbonate, carbonate, a zwitterionic buffer, morpholine propanesulphonic acid, dextrose, lactose, tartaric acid, and arginine; and from 5 mg/vial to 15 mg/vial of a caking agent or bulking agent selected from the group consisting of sodium chloride, gelatin, mannitol, inositol, sorbitol, polyethylene glycol, and polyvinyl pyrrolidone (PVP), wherein the composition is free of sequestering agent and wherein the composition is further radiolabeled with desired radioisotope.

In another aspect of the present invention, the method of preparing a two vial kit radiolabeled composition comprises: a) adding 1-10 mL ⁶⁸Ga in hydrochloric acid solution to vial one containing lyophilized composition; b) adding buffer solution of vial two to the vial one solution of step (a); c) heating the vial for 5-30 minutes at a temperature ranging from 60-120° C. and cooling for 2-10 minutes; and d) diluting the vial with saline at a volume of 4-14 ml, wherein the saline is sodium chloride present at a concentration of about 7 mg/mL-11 mg/mL.

In another aspect of the present invention, the method of preparing a two vial kit radiolabeled composition comprises: a) adding 1-10 mL ⁶⁸Ga in hydrochloric acid solution to vial one containing lyophilized composition; b) adding buffer solution of vial two to the vial one solution of step (a); c) heating the vial for 5-30 minutes at a temperature ranging from 60-120° C. and cooling for 2-10 minutes; d) diluting the vial with saline at a volume of 4-14 ml, wherein the saline is sodium chloride present at a concentration of about 7 mg/mL-11 mg/mL; wherein the edotreotide is present in the solution at a concentration of about 3 μg/mL-4 mg/mL.

In another embodiment, the compositions according to the present invention can be used for imaging, diagnosis, and treatment of neuroendocrine tumors and prostate cancers.

Each embodiment disclosed herein is contemplated as being applicable to each of the other disclosed embodiments. Thus, all combinations of the various elements described herein are within the scope of the invention.

This invention will be better understood by reference to the examples and experimental data, which follow, but those skilled in the art will readily appreciate that the specific experiments detailed are only illustrative of the invention as described more fully in the claims, which follow thereafter.

### EXAMPLES

### Example 1—Single Vial Pharmaceutical Composition of Edotreotide

| | | | | |
|---|---|---|---|---|
| | Compo- | Compo- | Compo- | Compo- |
| Ingredient | sition A | sition B | sition C | sition D |

| | | | | | | | | |
|---|---|---|---|---|---|---|---|---|
| Edotreotide Acetate | 50 | μg | 50 | μg | 50 | μg | 42 | μg |

| | | | | | | |
|---|---|---|---|---|---|---|
| Sodium Succinate | 28 | mg | — | — | 27 | mg |

| | | | | | |
|---|---|---|---|---|---|
| Sodium Ascorbate | — | 90 | mg | — | — |

| | | | | | |
|---|---|---|---|---|---|
| Sodium Acetate | — | — | 13 | mg | — |

| | | | | | | | | |
|---|---|---|---|---|---|---|---|---|
| Ascorbic Acid* | 3 | mg | 3 | mg | 3 | mg | 3 | mg |

| | | | | | | |
|---|---|---|---|---|---|---|
| Mannitol | 5 | mg | 5 | mg | — | — |

| | | | | | |
|---|---|---|---|---|---|
| Inositol | — | — | 10 | mg | — |

| | | | | | |
|---|---|---|---|---|---|
| Sodium Chloride | — | — | — | 10 | mg |

*Gentisic acid may also be used in the composition

Method of Preparing Single Vial Composition of Edotreotide:

Buffer was added to deoxygenated water for injection and mixed until complete dissolution. The anti-oxidant and the caking agent or bulking agent were added to the solution and mixed until complete dissolution. Edotreotide was added and the obtained solution was mixed and brought to the final quantum sufficit target with deoxygenated water for injection and then lyophilized.

Method of Preparing a Two-Vial Composition of Edotreotide:

Vial A—Stabilizer was added to deoxygenated water and mixed until complete dissolution. Caking agent or bulking agent was added to the solution and mixed until complete dissolution. Edotreotide was added and the solution was mixed for dissolution and brought to the final QS target with deoxygenated water for injection and then lyophilized.

Vial B—Buffer preparation—Sodium citrate 0.1M pH 5: 1 L of sodium citrate 0.1M was prepared by mixing 350 mL of citric acid 0.1M with 650 mL of sodium citrate 0.1M.

Radiolabeling Process for Single Vial Kit Composition with ⁶⁸Ga:

Method 1: Water (0.5 mL) was added to the freeze-dried vial. After about 1-2 minutes, Gallium-68 in hydrochloric acid solution was added. The vial was heated at a temperature of 95-105° C. for about 25 minutes and cooled for about 10 minutes, and then it was diluted to the desired volume with saline. Radiochemical Purity >95%

Method 2: Gallium-68 in hydrochloric acid solution was added to a freeze-dried vial. The vial was heated for 10 minutes at a temperature of 95-105° C. and cooled for about 10 minutes, and then it was diluted to the desired volume with saline. Radiochemical Purity >95%

Method 3: Water (0.5 mL) was added to the freeze-dried vial and the mixture was transferred into a microwave vial. After about 1-2 minutes, Gallium-68 in hydrochloric acid solution was added. The vial was exposed to microwave radiation for 10 minutes followed by 5 minutes of cooling. Radiochemical Purity >95%

### Example 2—Two Vial Pharmaceutical Composition of Edotreotide

| | Compo- | | Compo- | Compo- | | Compo- |
|---|---|---|---|---|---|---|
| | sition A | | sition A | sition B | | sition B |
| Ingredient | Vial A | | Vial B | Vial A | | Vial B |
| Edotreotide Acetate | 50 | μg | — | 50 | μg | — |
| Sodium Succinate | — | | 1.5 mL | — | | |
| Sodium Acetate | — | | — | — | | 1.2 mL |
| Ascorbic Acid* | 2.3 | mg | — | 3 | mg | — |

| | | | | | |
|---|---|---|---|---|---|
| Inositol | 10 | mg | — | — | — |

| | | | | | |
|---|---|---|---|---|---|
| Sodium Chloride | — | — | 10 | mg | — |

*Gentisic acid may also be used in the composition

Radiolabeling Process for Two Vial Kit Composition with Gallium-68

Method 1: Gallium-68 in hydrochloric acid solution was added to Vial A. After 1-2 minutes, buffer solution was added and the vial was heated for about 15 minutes and cooled for 10 minutes, and then it was diluted to the desired volume with saline. Radiochemical Purity >95%

Method 2: Buffer solution was added to Vial A. After 1-2 minutes, Gallium-68 in hydrochloric acid solution was added. The vial was heated for 15 minutes and cooled for about 10 minutes, and then it was diluted to the desired volume with saline. Radiochemical Purity >95%

Method 3: Gallium-68 in hydrochloric acid solution was mixed with the buffer solution, and the mixture was added to Vial A. The vial was heated for 15 minutes and cooled for about 10 minutes, and then it was diluted to the desired volume with saline. Radiochemical Purity >95% The radiochemical purity of the composition was determined and found to be not less than 95% for both single-vial and two-vial composition kits.

### Example 3—Edotreotide (DOTATOC) Single Lyophilized Vial (10/25 mL) Formulation (Composition E)

| Vial | | | Amount/ | | Specification | |
|---|---|---|---|---|---|---|
| format | Ingredient | Role | Vial | | range in the Vial | |
| 10/25 mL | Edotreotide | API | 50 | μg | 0.045-0.055 | mg/vial |
| | Acetate/ | Precursor | | | | |
| | Peptide | | | | | |
| | precursor | | | | | |
| | Sodium | Buffer | 67.9 | mg | 54.3-81.5 | mg/vial |
| | acetate | | | | | |
| | anhydrous | | | | | |
| | L-Ascorbic | Anti- | 3.0 | mg | 2.4-3.6 | mg/vial |
| | acid* | oxidant | | | | |
| | Sodium | Caking | 10 | mg | 8.0-12.0 | mg/vial |
| | chloride | agent or | | | | |
| | | Bulking | | | | |
| | | Agent | | | | |

*Gentisic acid may also be used in the composition

Single Vial Preparation Procedure:

All the manufacturing steps of the Edotreotide (DOTATOC) single vial formulation were performed under nitrogen (N₂) flow. Around 30 ml of ultrapure deoxygenated water (O₂ level <1.0 ppm) were added to a beaker equipped with a magnetic stir bar and the stirring was set to 250 rpm. Then 150 mg of ascorbic acid or gentisic acid, 3.395 g of sodium acetate, and 0.5 g of sodium chloride were then added to the solution. The pH of the bulk solution was measured using a pH meter. 3.22 mg of Edotreotide acetate was added to the bulk solution and the solution was stirred for 5 mins. The DOTATOC bulk solution was then QS to 50 ml with ultrapure water. The final pH was measured and the mixture was filtered and dispensed in 10 ml vials and lyophilized using the cycle provided in Table 1. After the lyophilization, the vials were sealed with aluminum seals. The lyophilization cycle used a New cycle of −32° C. primary drying. The analytical result of the formulation is given in Table 2:

TABLE 1
Thermal Treatment

| Temperature (° C.) | Time (Min) | Ramp/Hold (R/H) |
|---|---|---|
| −40 | 5 | H |
| −45 | 15 | R |
| −45 | 15 | H |

Primary Drying and secondary step

| Temperature (° C.) | Time (Min) | Vacuum | Ramp/Hold (R/H) |
|---|---|---|---|
| −45 | 5 | 30 | H |
| −32 | 120 | 30 | R |
| −32 | 600 | 30 | H |
| −32 | 600 | 30 | H |
| 40 | 490 | 30 | R |
| 40 | 360 | 30 | H |
| 20 | 20 | 30 | R |
| 20 | 600 | 30 | H |

Analytical Results:

TABLE 2

| Test | Results | |
|---|---|---|
| Description | Homogeneous powder/cake | |
| DOTATOC Assay | 0.054 | mg/vial |
| Sodium Acetate | 66.7 ± 0.42 | mg/vial |

Radiolabeling Procedure with ⁶⁸Ga:

A ⁶⁸Ge/⁶⁸Ga generator radioisotope generator was eluted with 0.1N HCl (1, 3, 4, or 5 ml) directly to the Edotreotide vial kit equipped with a venting needle and placed in an appropriate lead pot. After the elution, the vial was gently shaken and the activity was measured in the calibrated dose calibrator. The vial was incubated for 30 min at 95° C. in the digital heating block, after which it was cooled down for 10-15 min at room temperature and the pH was measured.

Quality Control:

The labelling efficiency of [⁶⁸Ga]Ga-DOTATOC was determined using iTLC-SG paper as the heat activated stationary phase pre-cut to 11.5×1.0 cm. The start line was marked at 2 cm and the solvent front was marked at 11 cm. The 50/50 v/v mixture of ammonium acetate (1M) and methanol was used as the mobile phase, which was placed in the glass tubes. One 1 μL of the mixture was spotted at the start line and the strip was immediately put for migration. The developed strips were air-dried and analyzed using the calibrated Bioscan using an appropriate method. The [68Ga]Ga-Edotreotide (DOTATOC) migrates with the solvent front, while free Ga-68 ions and hydrolyzed Ga-68 colloids remain at the start line. The radiochemical purity must be not less than 95%. The results are summarized in Table 3:

TABLE 3
Table 3. Radiolabelling results with different volumes
and activity of Ga-68 chloride solution:

| Ga-68 | Ga-68 | | |
|---|---|---|---|
| volume | activity | pH after | Radiochemical |
| used (ml) | used (mCi) | radiolabeling | Purity (%) |
| 1 | 5 | 5.5-6.0 | 97.03 ± 0.60 |
| 3 | 10 | 5.0-5.5 | 97.37 ± 0.54 |
| 4 | 15 | 5.0-5.5 | 97.72 ± 0.26 |
| 4 | 15 | 5.0 | 98.61 ± 0.18 |
| 5 | 25 | 5.0 | 98.68 ± 0.20 |

### Example 4—Edotreotide (DOTATOC) Single Lyophilized Vial Formulation (Composition F)

| Vial | Ingredient | Role | Amount/Vial | |
|---|---|---|---|---|
| 30 mL | EdotreotideAcetate | API Precursor | 0.045-0.055 | mg/vial |
| | Sodium acetate | Buffer | 54.3-81.5 | mg/vial |
| | anhydrous | | | |
| | L-Ascorbic acid* | Antioxidant | 2.4-3.6 | mg/vial |
| | Sodium chloride | Caking agent or | 8.0-12.0 | mg/vial |
| | | Bulking Agent | | |

*Gentisic acid may also be used in the composition

Kit Preparation Procedure: Process is similar to the process mentioned in Example 3

The lyophilization cycle used: New cycle −32° C. primary drying is employed as provided in Table 4:

TABLE 4
Thermal Treatment

| Temperature (° C.) | Time (Min) | Ramp/Hold (R/H) |
|---|---|---|
| −40 | 5 | H |
| −45 | 15 | R |
| −45 | 15 | H |

Primary drying and secondary step

| Temperature (° C.) | Time (Min) | Vacuum | Ramp/Hold (R/H) |
|---|---|---|---|
| −45 | 5 | 30 | H |
| −32 | 120 | 30 | R |
| −32 | 600 | 30 | H |
| −32 | 600 | 30 | H |
| 40 | 490 | 30 | R |
| 40 | 360 | 30 | H |
| 20 | 20 | 30 | R |
| 20 | 600 | 30 | H |

The analytical results along with the physico-chemical parameters are mentioned below in Table 5.

TABLE 5

| TEST | RESULTS | |
|---|---|---|
| Edotreotide Assay | 0.049 | mg/vial |
| Sodium Acetate | 61.9 | mg/vial |
| Oxygen Content | 0.2% | |
| Sodium Chloride | 10.0 | mg/vial |
| Antioxidant Content | 3.0 | mg/vial |

After reconstitution
With 1.1 mL of 0.1N HCl + 12.9 mL NaCl

| | | |
|---|---|---|
| Edotreotide Assay | 3.64 | mg/mL |
| Sodium Chloride | 8.7 | mg/mL |

| | |
|---|---|
| pH | 6.0 |

With 4 mL of 0.1 N HCl + 10 mL NaCl

| | | |
|---|---|---|
| Edotreotide Assay | 3.82 | mg/mL |
| Sodium Chloride | 8.7 | mg/mL |

| | |
|---|---|
| pH | 4.7 |

With 5 mL of 0.1N HCl + 9 mL NaCl

| | | |
|---|---|---|
| Edotreotide Assay | 3.92 | mg/mL |
| Sodium Chloride | 8.6 | mg/mL |

| | |
|---|---|
| pH | 4.5 |

Radiolabeling Procedure with ⁶⁸Ga:

A ⁶⁸Ge/⁶⁸Ga generator was eluted with ultrapure 0.1N HCl (5 ml) directly to the Edotreotide vial kit equipped with a venting needle and placed in an appropriate lead pot. After the elution, the vial was gently shaken and the activity was measured in the calibrated dose calibrator. The vial was incubated for 30 min at 95° C. in the digital heat block designed for a 30 mL vial, after which it was cooled down for 10 min at room temperature. 0.9% saline (9 ml) was added to the vial to complete the volume to 14 ml. The pH was measured using pH strips.

Quality Control:

The labelling efficiency of [⁶⁸Ga]Ga-Edotreotide (DOTATOC) was determined using iTLC-SG paper as the heat activated stationary phase pre-cut to 11.5×1.0 cm. The start line was marked at 2 cm and the solvent front was marked at 11 cm. The 50/50 v/v mixture of ammonium acetate (1M) and methanol was used as the mobile phase, which was placed in the glass tubes (1 ml per tube). 1 μL of the mixture was spotted at the start line and the strip was immediately put for migration. The developed strips were air-dried and analyzed using the calibrated Bioscan using an appropriate method. The [⁶⁸Ga]Ga-Edotreotide (DOTATOC) migrates with the solvent front, while free Ga-68 ions and hydrolyzed Ga-68 colloids remain at the start line. The radiochemical purity must be not less than 95% as mentioned herein Table 6:

TABLE 6
Radiolabeling results:

| Ga-68 activity used, | pH after | |
|---|---|---|
| mCi | radiolabelling | RCP, % |
| 9 mCi | 4.5-5.0 | 97.83 ± 0.25 |

Conclusion: The single vial composition E and composition F of Example 3 and Example 4 respectively comprise only one stabilizer/antioxidant at an amount of 3.0 mg/vial, which shows unexpectedly satisfactory results in view of the stability of the composition. The test results like Edotreotide (DOTAOC) assay and others represent that the values are within the acceptable range and the overall formulations are stable by using only one stabilizer/antioxidant, wherein the compositions are free of sequestering agent.

Examples of Labeling with ¹⁷⁷Lu and ²⁵⁵ Ac:

Radiolabeling procedure for ['⁷⁷Lu]Lu-Edotreotide (DOTATOC):

Insert 16G cannula to Lu-177 C13 vial and draw the complete activity using a syringe and check the activity. Add ˜200 mCi of Lu -177Cl₃ solution to Edotreotide (DOTATOC) using a syringe. Check the pH using pH strips (should be 4-8). Shake well for dissolution and heat the vial for 30 minutes at 90° C. After heating, place the vial B in a lead container for cooling for 10 minutes.

Quality Control by Radio-TLC:

Spot one drop on a Whatman 3MM blotting paper (10 cm×1 cm) and develop in 1 mL of acetonitrile and water (1:1 ratio) in the chromatography chamber. Dry the paper and read the paper using bioscan. Radiochemical Purity >90%.

Radiolabeling procedure for [²⁵⁵ Ac]Ac-Edotreotide (DOTATOC):

Add aliquot of ²²⁵AC buffered stock solution and add to Edotreotide DOTATOC vial. Heat 95° C., 5 min, microwave; Add DTPA solution (10-20 μL of 10 mM DTPA) to react with free metal ions (including unbound ²²⁵Ac); Take 1-10 μL aliquot for reaction yield determination by iTLC.

Reversed Phase (SepPak) Purification:

Condition a reversed phase sep-pak cartridge: Add reaction mixture to the cartridge; Rinse with appropriate solution/solvent (H₂O) to eliminate all free metals (including those bound with DTPA); Eluate Labeled DOTATOC with ethanol. Dilute with 0.9% NaCl.

Quality Control by Radio-TLC:

Determine radiochemical by radio-TLC analysis (10 μL, iTLC-SG as a stationary phase, 10 mM EDTA or 10 mM DTPA as a mobile phase). [²²⁵Ac]Ac-Edotreotide (DOTATOC) will remain at the baseline (Rf=0) and any ²²⁵AC that has de-complexed from the chelate will travel with the solvent front (Rf=1). Radiochemical Purity >95%.

What is claimed:

1. A pharmaceutical cold kit composition comprising:
a chelating agent linked to somatostatin receptor binding organic moiety;
a stabilizer, wherein the stabilizer is selected from the group consisting of ascorbic acid, sodium bisulfite, gentisic acid, glutamate, metabisulfite, monothioglycerol, propyl gallate, sulfite sodium;
a buffer; and
a caking agent or bulking agent,
wherein the composition is free of sequestering agent and wherein the composition is further radiolabeled with desired radioisotope.

2. A pharmaceutical cold kit composition comprising:
edotreotide or its pharmaceutically acceptable salt thereof;
a stabilizer selected from the group consisting of ascorbic acid, sodium bisulfite, gentisic acid, glutamate, metabisulfite, monothioglycerol, propyl gallate, sulfite sodium, tocopherol alpha, thioglycolate, formaldehyde sulfoxylate sodium, melatonin;
a buffer selected from the group consisting of sodium acetate, sodium succinate, tartrate, citrate, formate, HEPES buffer, lactate, TRIS, phosphate, nitrate, phosphate, borate, bicarbonate, carbonate, a zwitterionic buffer, morpholine propane sulphonic acid, dextrose, lactose, tartaric acid, and arginine, and combinations thereof; and
a caking agent or bulking agent selected from the group consisting of sodium chloride, gelatin, mannitol, inositol, sorbitol, polyethylene glycol, and polyvinyl pyrrolidone (PVP), and combinations thereof;
wherein the composition is free of any sequestering agent and wherein the composition is further radiolabeled with desired radioisotope.

3. The pharmaceutical composition according to claim 1, wherein the chelating agent is selected from the group consisting of DOTA, TETA, NOTA, DTPA, NTA, EDTA, DO3A, NOC, DOTAGA, HBED, HBECC, NODAGA, DFO, EDTA, 6SS, B6SS, PLED, TAME, YM103, and H2dedpa.

4. The pharmaceutical composition according to claim 1, wherein the composition is a single vial or multiple vial kit.

5. The pharmaceutical composition according to claim 4, wherein the multiple vial kit is selected from the group consisting of two vials, three vials, or four vials.

6. The pharmaceutical composition according to claim 1, wherein the buffer is selected from the group consisting of sodium acetate, sodium succinate, tartrate, citrate, formate, and combinations thereof.

7. The pharmaceutical composition according to claim 1, wherein the pH of the composition ranges from about 3 to 8.

8. The pharmaceutical composition according to claim 5, wherein the two vial kit comprises:
a) a first vial having a vial size of 1-30 mL comprising a chelating agent linked to somatostatin receptor binding organic moiety, a stabilizer, and a caking agent or bulking agent; and
b) a second vial comprising a buffer.

9. The pharmaceutical composition according to claim 5, wherein the three vial kit comprises:
a) a first vial having a vial size of 1-30 mL and comprising a chelating agent linked to somatostatin receptor binding organic moiety, a stabilizer, and a caking agent or bulking agent;
b) a second vial comprising a buffer; and
c) a third vial comprising a hydrochloric acid solution.

10. The pharmaceutical composition according to claim 1, wherein composition is radiolabeled with a radioisotope selected from copper-64, gallium-68, gallium-67, gallium-66, lutetium-177, yttrium-86, yttrium-90, technetium -99, indium-114, indium-111, scandium-47, scandium-44, scandium-43, zirconium-89, bismuth-213, bismuth-212, actinium-225, lead-212, rhenium-188, rhenium-186, and rubidium-82.

11. The pharmaceutical composition according to claim 10, wherein the radioisotope is gallium-68 or lutetium-177.

12. The pharmaceutical composition according to claim 10, wherein the initial radiochemical purity of radiolabeled composition is at least 95 percent.

13. The pharmaceutical composition according to claim 10, wherein the initial radiochemical purity of radiolabeled composition is at least 97 percent.

14. The pharmaceutical composition according to claim 10, wherein the initial radiochemical purity of radiolabeled composition is at least 99 percent.

15. The radiolabeled composition according to claim 10, wherein the composition comprises less than 3 percent of gallium-68 in colloidal form.

16. The radiolabeled composition according to claim 10, wherein the composition comprises less than 2 percent of gallium-68 (III) iron.

17. A method for preparing a single vial kit of radiolabeled composition according to claim 11, the method comprising:
a) adding 0.5 mL of water to a lyophilized vial;
b) adding gallium-68 in hydrochloric acid solution to the solution of step (a) after 1-2 minutes;
c) heating the vial for 5-30 minutes at a temperature ranging from 60-120° C. and cooling for 2-10 minutes; and
d) diluting the vial with saline at a volume of 4-14 mL, wherein the saline is sodium chloride present at a concentration of about 7 mg/mL-11 mg/mL;
wherein the edotreotide is present in the solution at a concentration of about 3 μg/mL-4 mg/mL.

18. A reconstituted pharmaceutical composition comprising:
(a) edotreotide or its pharmaceutically acceptable salt thereof;
(b) a stabilizer selected from the group consisting of ascorbic acid, sodium bisulfite, gentisic acid, glutamate, metabisulfite, monothioglycerol, propyl gallate, sulfite sodium, tocopherol alpha, thioglycolate, formaldehyde sulfoxylate sodium, histidine, and melatonin;
(c) a buffer selected from the group consisting of sodium acetate, sodium succinate, tartrate, citrate, formate, HEPES buffer, lactate, TRIS, phosphate, nitrate, phosphate, borate, bicarbonate, carbonate, a zwitterionic buffer, morpholine propanesulphonic acid, dextrose, lactose, tartaric acid, and arginine and combinations thereof; and
(d) a caking agent or bulking agent selected from the group consisting of sodium chloride, gelatin, mannitol, inositol, sorbitol, polyethylene glycol, and polyvinyl pyrrolidone (PVP) and combinations thereof,
wherein the composition is free of sequestering agent and wherein the composition is further radiolabeled with desired radioisotope and the initial radiochemical purity of radiolabeled composition is at least 95 percent.

19. A reconstituted pharmaceutical composition comprising:
(e) from 0.045 mg/vial to 0.055 mg/vial of edotreotide or its pharmaceutically acceptable salt thereof;
(f) from 2 mg/vial to 5 mg/vial of a stabilizer selected from the group consisting of ascorbic acid, sodium bisulfite, gentisic acid, glutamate, metabisulfite, monothioglycerol, propyl gallate, sulfite sodium, tocopherol alpha, thioglycolate, formaldehyde sulfoxylate sodium, histidine, and melatonin;
(g) from 45 mg/vial to 90 mg/vial of a buffer selected from the group consisting of sodium acetate, sodium succinate, tartrate, citrate, formate, HEPES buffer, lactate, TRIS, phosphate, nitrate, phosphate, borate, bicarbonate, carbonate, a zwitterionic buffer, morpholine propanesulphonic acid, dextrose, lactose, tartaric acid, and arginine and combinations thereof; and
(h) from 5 mg/vial to 15 mg/vial of a caking agent or bulking agent selected from the group consisting of sodium chloride, gelatin, mannitol, inositol, sorbitol, polyethylene glycol, and polyvinyl pyrrolidone (PVP) and combinations thereof,
wherein the composition is free of sequestering agent and wherein the composition is further radiolabeled with desired radioisotope and the initial radiochemical purity of radiolabeled composition is at least 95 percent.
